(19) United States
(12) Patent Application Publication
(10) Pub. No.: US 2011/0146835 A1
(43) Pub. Date: Jun. 23, 2011

(54) AUTOMATED PHARMACY DRUG HANDLING AND PRESCRIPTION VERIFICATION SYSTEM AND METHOD

(76) Inventor: Robert Terzini, Corinth, TX (US)

(21) Appl. No.: 13/060,255

(22) PCT Filed: Aug. 23, 2009
(86) PCT No.: PCT/US2009/054712

§ 371 Date: Feb. 22, 2011

Related U.S. Application Data
(63) Continuation-in-part of application No. 12/396,417, filed on Mar. 2, 2009.
(60) Provisional application No. 61/091,345, filed on Aug. 23, 2008.
(60) Provisional application No. 61/112,776, filed on Nov. 10, 2008.
(60) Provisional application No. 61/114,462, filed on Nov. 14, 2008.

(51) Int. Cl. B65B 1/04

(52) U.S. Cl. 141/1; 141/98

(57) ABSTRACT

An intake to exit security system for high-volume pharmacies provides maximum security from tampering and assures accuracy. The system immediately assigns bar codes to shipments upon arrival and then tracks them through warehousing, bulk distribution, prescription dispensing and shipping to patients, hospitals and drugstores. Bar-coded lock neck devices secure bulk drug canisters to bar-coded dispensing machines at specified dispensing stations where the machines dispense drugs into pre-labeled prescription bottles according to prescription indicia on the labels. Bottles then undergo content analysis and certification before packaged and shipped to customers. A Ramon laser spectral analysis contrasts the bottle contents to a library of known spectral signatures of drugs, and the pharmacist is alerted to any detected difference. A simultaneously captured visual image of the pills enables the pharmacist visually to compare the contents to a library of known visual appearances of the drugs. Both analyses are recorded for prescriptions certified and forwarded to customers. Deviations are excised without disrupting flow of other prescriptions, and the system automatically reassigns an incorrectly filled prescription to another bottle which starts anew through the system. Full bottles of commonly used drugs and specialized containers for irregularly shaped objects, creams and ointments may be pre-filled and inventoried for later collation with prescription bottles at the packaging and shipping stage.

### BACKGROUND OF THE INVENTION

1. Field of the Invention

This invention relates generally to automated prescription filling systems and particularly to apparatus and methods for maintaining security of drugs and prescription filling processes. More particularly, this invention relates to a system and method for securely receiving and warehousing drugs, and for dispensing prescriptions and verifying their accuracy with minimal or no manual intervention.

2. Description of Related Art

Automated pharmaceutical prescription-filling systems answer a need for high-volume pharmaceutical deliveries. Coupled with the use of mail order delivery service, automated, central filling of prescriptions has been highly successful in lowering costs of providing drugs to consumers. Benefits include increased volume, lower costs, reduction of pharmacy personnel, inventory control, substance control, automated documentation, and quick turn-around times. Equally importantly, such systems assume most of the drudgery and relieve professional pharmacists from the tedium and fatigue of monitoring a multitude of high-volume orders, thereby reducing rates of medication errors.

Though largely automated, many prescription filling systems require manual intervention whereby container security of drugs may be compromized. For example, though automatic pill counters increase accuracy, they typically cannot assure that the pills in them indeed are what the system thinks they are. Such machines usually employ a hopper into which pills from drug supplier containers must be transferred, a process that remains vulnerable both to theft and contamination. A need exists for a system and method for handling drugs in a pharmacy that is secure all the way from receiving to shipping.

In all legitimate pharmacies, a licensed pharmacist ultimately is responsible for prescription accuracy. In automated prescription filling systems, however, the sheer volume of patient prescriptions being filled hourly threatens to exceed even the most diligent pharmacist's fatigue and attention levels. Means for assisting pharmacists in verifying accuracy of prescriptions after they have been dispensed should not require opening the containers and exposing the prescriptions to tampering, and should alert the pharmacist of possible errors rather than depend upon him to recognize and interdict them by himself.

### SUMMARY OF THE INVENTION

An intake to exit security system for high-volume pharmacies provides maximum security from tampering and assures accuracy. The system immediately assigns bar codes to shipments upon arrival and then tracks them through warehousing, bulk distribution, prescription dispensing and shipping to patients, hospitals and drugstores. Bar-coded lock neck devices secure bulk drug canisters to bar-coded dispensing machines at specified dispensing stations where the machines dispense drugs into pre-labeled prescription bottles according to prescription indicia on the labels. Bottles then undergo content analysis and certification before being packaged and shipped to customers. A Ramon laser spectral analysis contrasts the bottle contents to a library of known spectral signatures of drugs, and the pharmacist is alerted to any detected difference. A simultaneously captured visual image of the pills enables the pharmacist visually to compare the contents to a library of known visual appearances of the drugs. Both analyses are recorded for prescriptions certified and forwarded to customers. Deviations are excised without disrupting flow of other prescriptions, and the system automatically reassigns an incorrectly filled prescription to another bottle which starts anew through the system. Full bottles of commonly used drugs and specialized containers for irregularly shaped objects, creams and ointments may be pre-filled and inventoried for later collation with prescription bottles at the packaging and shipping stage.

### BRIEF DESCRIPTION OF THE DRAWINGS

The novel features believed characteristic of the present invention may be set forth in appended claims. The invention itself, however, as well as a preferred mode of use and further objects and advantages thereof, will best be understood by reference to the following detailed description of an illustrative embodiment when read in conjunction with the accompanying drawings, wherein:

FIG. 1 shows in quartering perspective view an automated prescription filling system utilizing the prescription container filling system of the present invention.

FIG. 2 depicts the automated prescription filling system of FIG. 1 in top plan view.

FIGS. 3A-3C and 3H detail a preferred embodiment of the prescription containers used in the prescription filling system of FIG. 1.

FIGS. 3D-3F detail an alternate embodiment of the prescription containers used in the prescription filling system of FIG. 1.

FIG. 3G details a package for shipping the prescription containers used in the prescription filling system of FIG. 1.

FIG. 4 shows in side elevational view one channel of the automated prescription filling system of FIG. 1, with the container filling apparatus of the present invention.

FIG. 5 shows in quartering perspective view the lower portion of the prescription container filling stage of the of the present invention, partially cut-away to reveal the container transport table and automatic closure and sealing apparatus inside.

FIGS. 6A, 6B show a bulk pharmaceutical canister and lock neck locking device used with the filling stage of FIG. 5.

FIGS. 7A, 7B depict in quartering perspective views of the front and back, respectively, of a dispensing machine with the canister and lock neck of FIGS. 6A, 6B installed.

FIG. 8 shows a schematic of one of the pharmaceutical dispensing machines of FIG. 5, including the process by which containers are matched to and filled with pharmaceuticals.

FIG. 9 depicts a pharmacy management system monitoring screen showing the dispensing station of FIG. 5 in operation.

FIGS. 10A, 10B depict a prescription certification station of the present invention.

FIG. 11 shows a laser spectral analysis machine used in the prescription certification station of FIGS. 10A, 10B.

FIG. 12 is a schematic diagram of the functioning parts of the laser spectral analysis machine device of FIG. 11.

FIGS. 13A-13D demonstrate an autofocus feature of the laser machine of FIG. 11 in different states of focus on a bottle for pharmaceuticals.

FIG. 14 depicts a pharmacist's verification screen for utilizing and comparing the information derived from the laser spectral analysis machine device of FIGS. 11, 12.

FIG. 15 shows a flow chart and schematic overview of the system of FIG. 1.

FIG. 16 shows a flow chart and schematic of the process by which shipments from drug manufacturers are handled and stored upon receipt.

FIG. 17 shows a flow chart and schematic of the process by which a new product is introduced into the system of FIG. 1.

FIG. 18 shows a flow chart and schematic of the process by which a canister of FIG. 6A is filled with pharmaceuticals.

FIG. 19 shows a flow charts and schematic of the steps to remove a dispenser used in the dispensing station of FIGS. 5-8.

FIGS. 20, 21 are flow charts and schematics of the steps to install and remove, respectively, the locked canister of FIGS. 6A, 6B onto and from the dispenser of FIGS. 7A, 7B

FIG. 22 shows a flow chart and schematic of the steps in the process of delivering containers to the dispensing station of FIG. 5.

FIG. 23 shows a flow chart and schematics of the steps in the process of dispensing pharmaceuticals in the dispensing station of FIG. 5.

FIG. 24 shows a flow chart and schematic of the steps in the process of the prescription fill certification and verification process.

### DESCRIPTION OF A PREFERRED EMBODIMENT

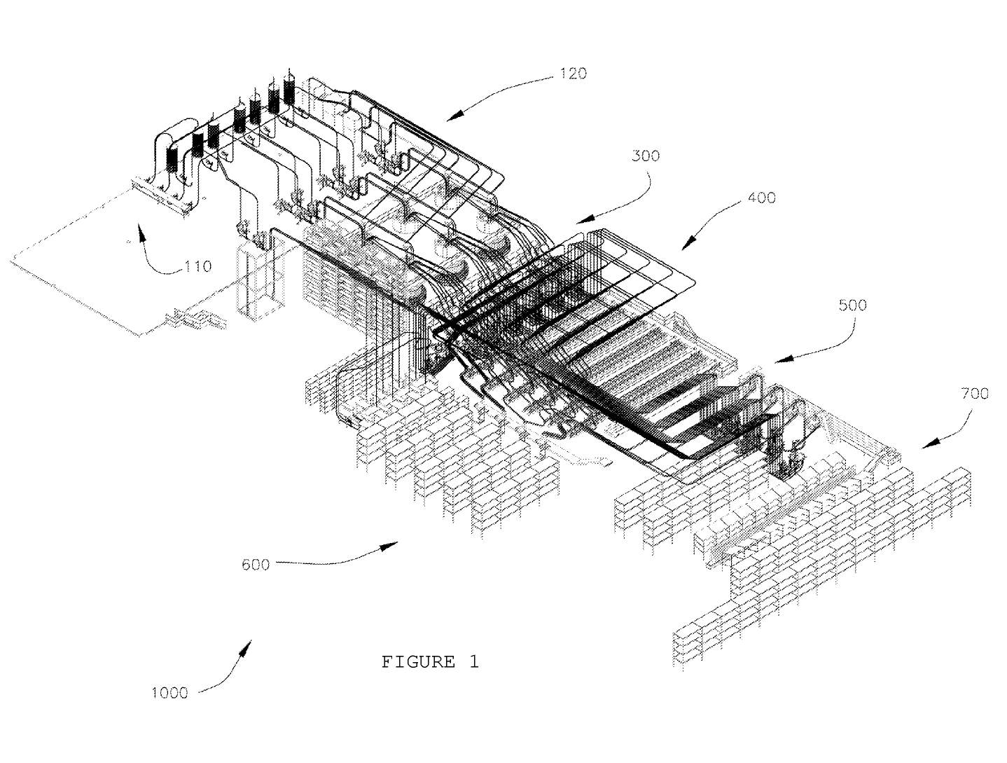

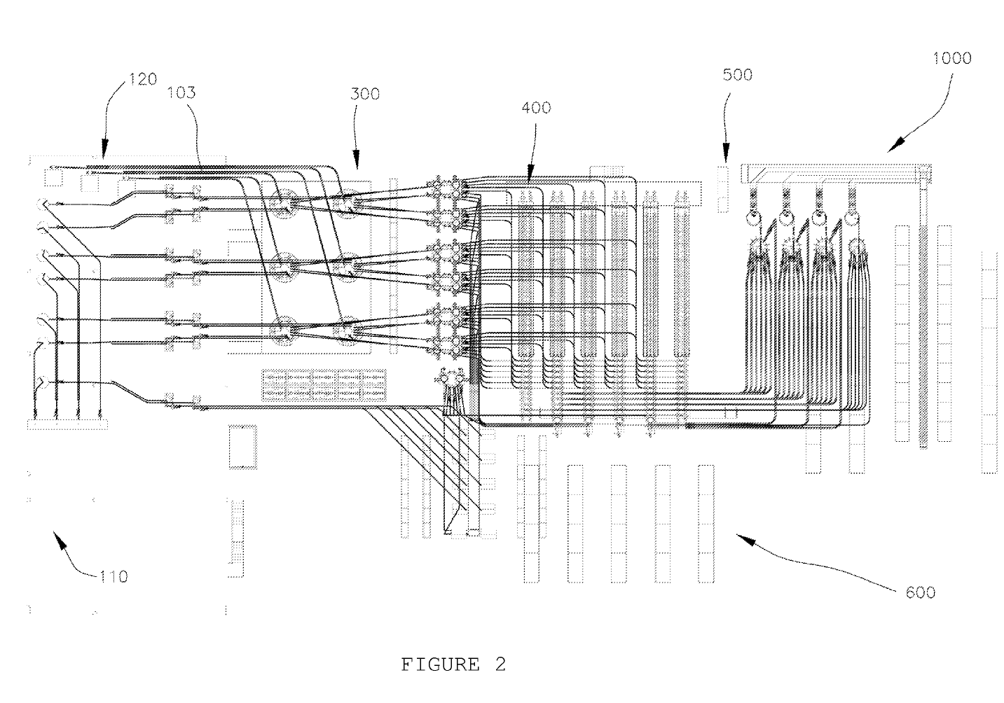

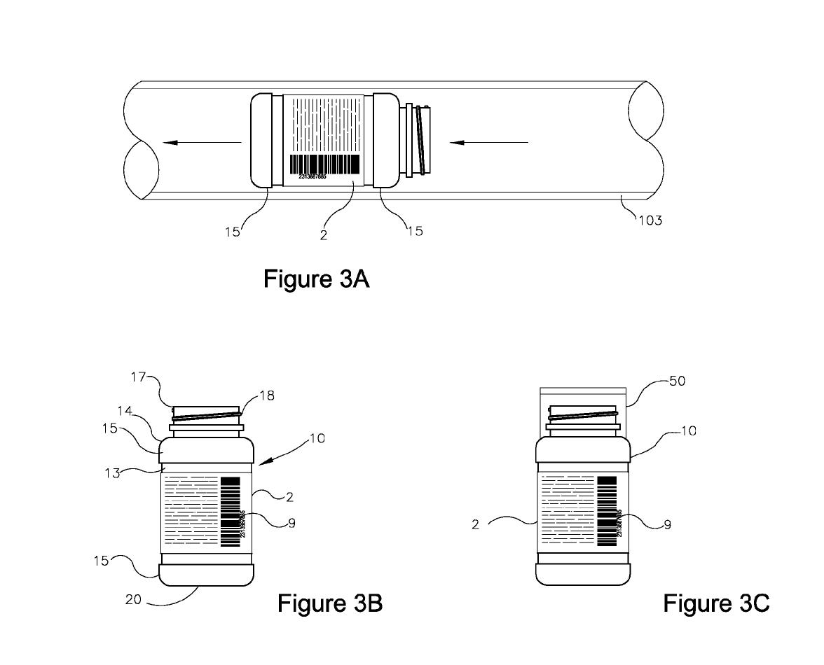

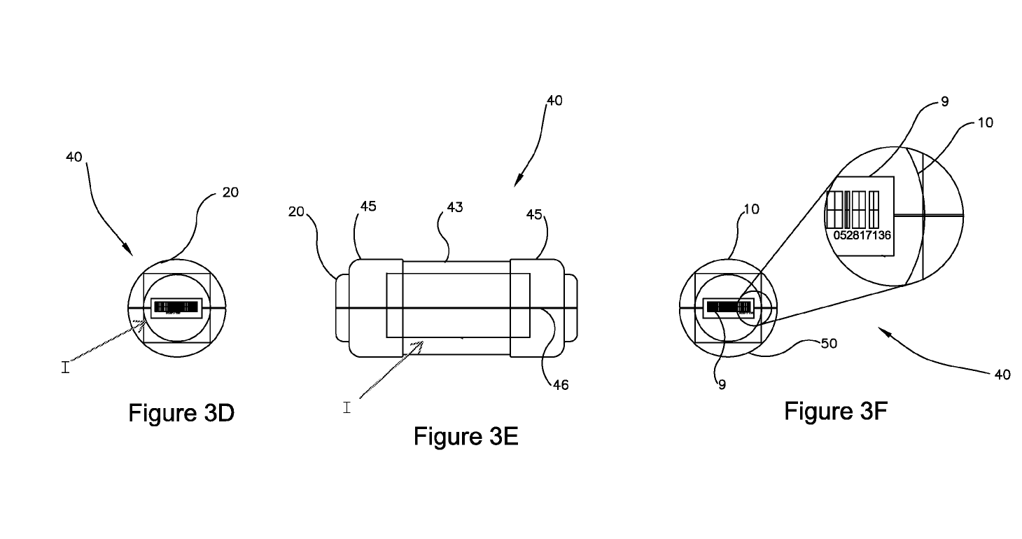

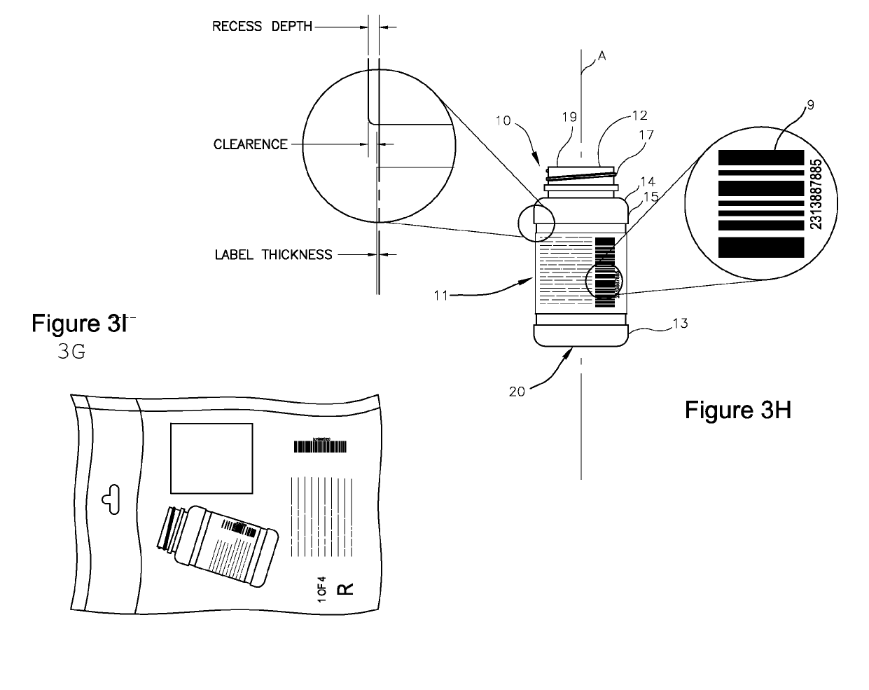

Referring now to the figures, and particularly to FIGS. 1-2, automated prescription filling system 1000 comprises prescription dispensing apparatus 300 feeding filled prescription containers 10 (see FIG. 3A) through prescription verification stage 400 and sortation conveyor system 500 to bagging, packaging and shipping system 600 where filled prescriptions are conveyed through common carriers to pharmacies, hospitals and individual patients (collectively “customers”). Apparatus 300 comprises a stage where containers 10 are filled according to each individual prescription from an array of individual pharmaceutical dispensing machines 200 and sealed by automated closure system 160. Bottles 10 then are transported to verification stage 400 where a pharmacist confirms that each container 10 contains the pharmaceutical required, then to conveyor 500 where container 10 is collected with other containers 10 for the same customer before being packaged at stage 600 and shipped, all without requiring human hands to handle containers 10 or their pharmaceutical contents.

NOTE: hereinafter, the present invention is discussed in large part in the context of a preferred container 10 embodiment utilizing prescription bottles, though a discussion of an alternate embodiment container 10 appears herein below. One having ordinary skill in the art will recognize, too, that other types of containers having similar features may be substituted and still considered to be within the spirit and scope of the present invention. One having ordinary skill in the art will recognize that, hereinafter where appropriate, reference to bottles 10 or containers 10 may mean either embodiment unless indication appears to the contrary. Further, it will be recognize that capping system 160 and 330 as described herein will be altered as needed to accommodate containers 10 which are not bottles or which utilize different closure and sealing means.

### Prescription Containers and Container Induction, Labeling and Transport

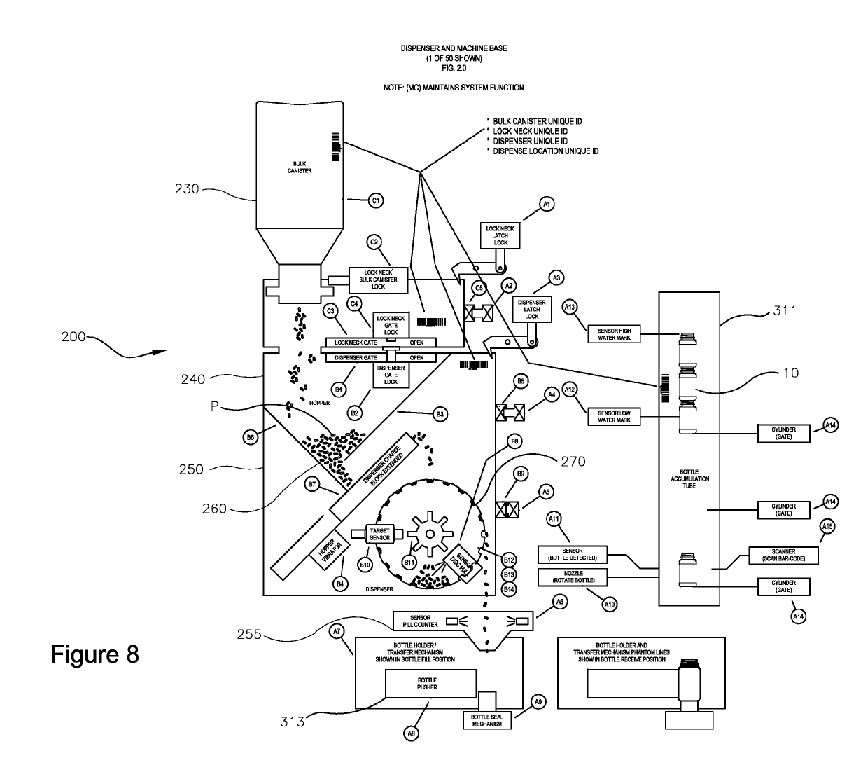

Turning first to FIGS. 3A-3C, a preferred embodiment of container 10 comprises a bottle having a regular, generally cylindrical cross section composed of walls 11 surrounding and concentric about longitudinal axis A and defining interior 12 into which a plurality of pharmaceuticals P (see FIG. 8) are introduced by dispensers 200. Bottle 10 is closed at bottom 20 opposite shoulders 14 where it reduces to neck 17 bearing threads 18 adapted to mate with a cylindrical cap 50 which closes and seals bottle 10. Though larger than neck 17, cap 50's diameter remains slightly smaller than that of walls 11 to remain within the profile of bottle 10 to pass through tubes 103.

FIGS. 3D-3F depict an alternate embodiment container 40 to bottle 10 comprises cylindrical body 41 having annular rings 45 disposed on each end and defining annular recess 43 in between. Unlike bottle 10, container 40 has no threads nor separate cap 50 to attach after filling at dispensing station 300. Instead, container 40 opens lengthwise at mouth 46 to provide access to its interior for insertion of irregularly shaped pharmaceutical products I such as vials of ointment, bottles of liquid drugs, sponges, wipes or the like or pharmaceuticals P so seldom dispensed that they do not justify dedicating a dispenser 200 to them, all of which also may be needed by the customer. Containers 40 will be sorted together with bottles 10 in sortation system 500. Hereinafter, reference to container 10 includes container 40 unless the context dictates otherwise, whereas reference to bottle 10 is limited to the bottles depicted in FIGS. 3A-3C, 3H.

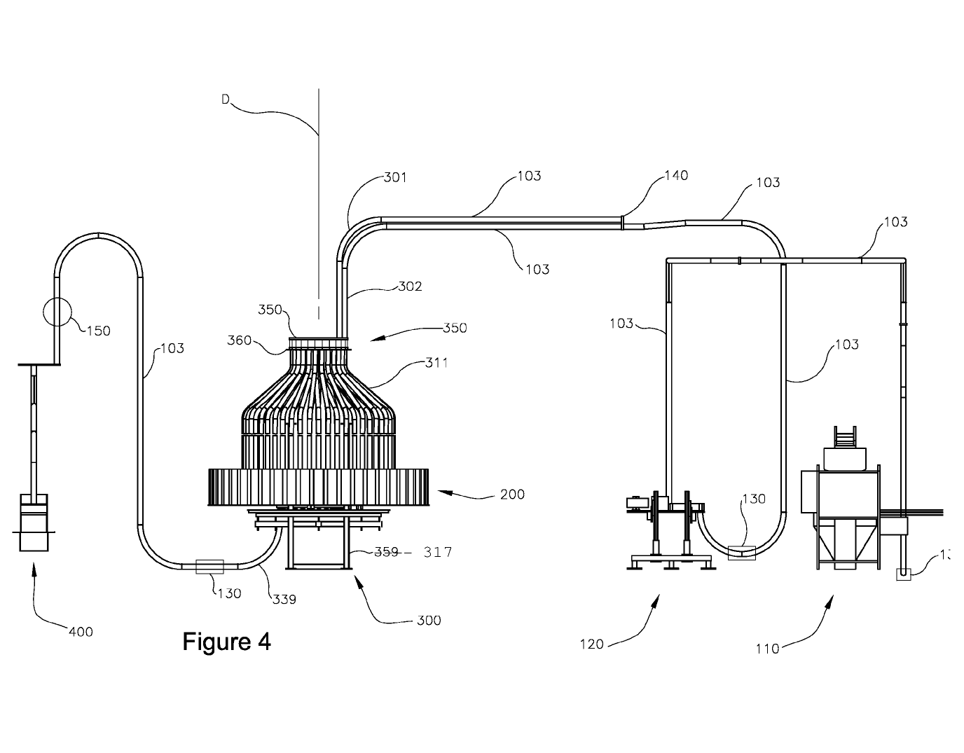

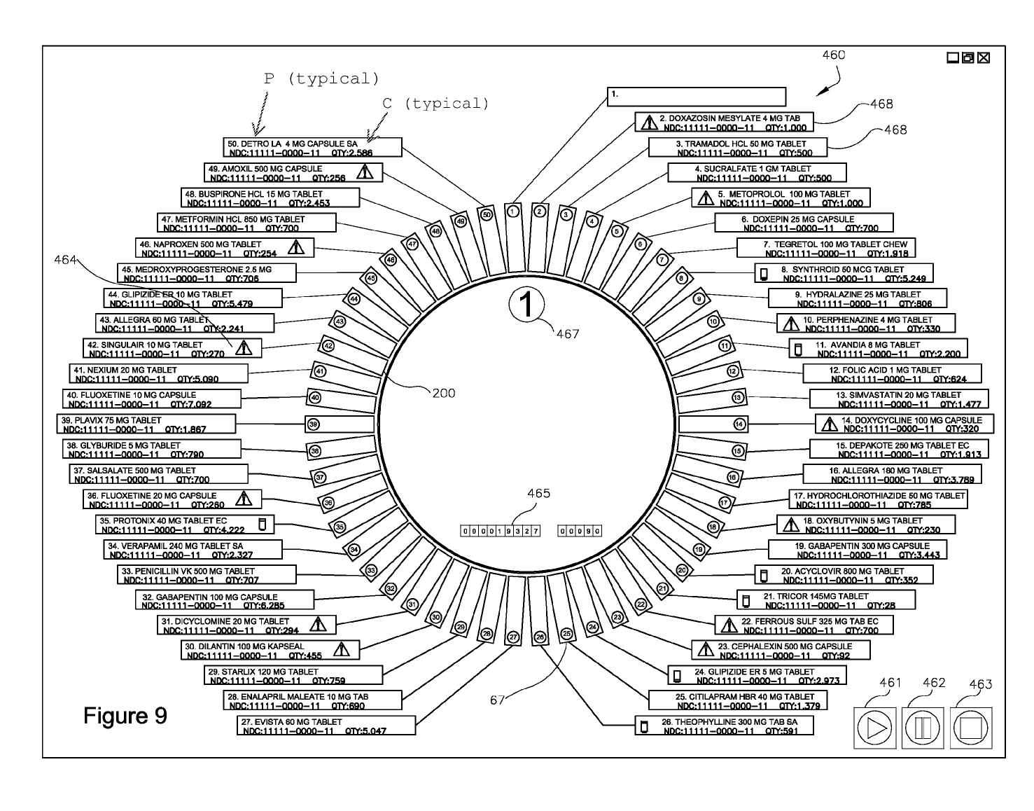

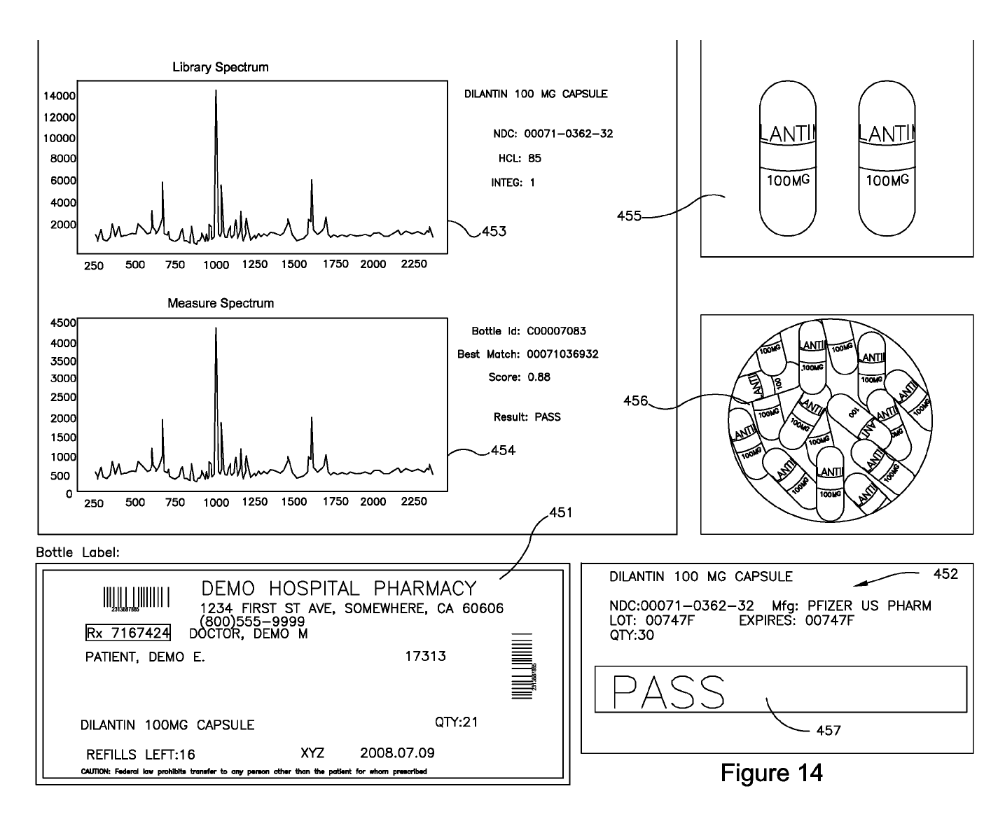

Disposed within annular recess 13, label 2 bears indicia 9, comprising a bar code or other machine readable encoding, adapted to inform prescription filling system 1000 and its various sensors and software (see FIGS. 9, 14 and discussion thereof hereinbelow), through use of a dynamically populated database, of the contents and expected location of container 10 within prescription filling system 1000. Container 10 is adapted to move, bottom 20 first, through pneumatic tubing 103 (FIG. 3A) between the various stages of system 1000. Impellers 130 (FIG. 4) disposed at the beginning of each run of tubes 103, provide impetus to move bottles 10 through tubes 103 between stages.

It will be understood that bottles 10 enter system 1000 uncapped, and that caps 50 must be placed on bottles 10 to seal them after they have been filled by dispensers 200 within stage 300. Bottles 10 are manufactured separately in bulk and inducted into system 1000 at unscrambler 110 which reorients them all facing the same direction and conveys them to labeling machines 120. Labelers 120 print labels 2, applies them to annular recesses 13, and then sends bottles 10 on to pharmaceutical dispensing system 300 for filling. Labels 2 carry indicia of the content and quantity of the pharmaceutical to be dispensed into bottle 10, and once bottle 10 receives label 2, prescription filling system 1000 tracks the prescription for said customer by following the location and status of each bottle 10.

### Dispensing Station

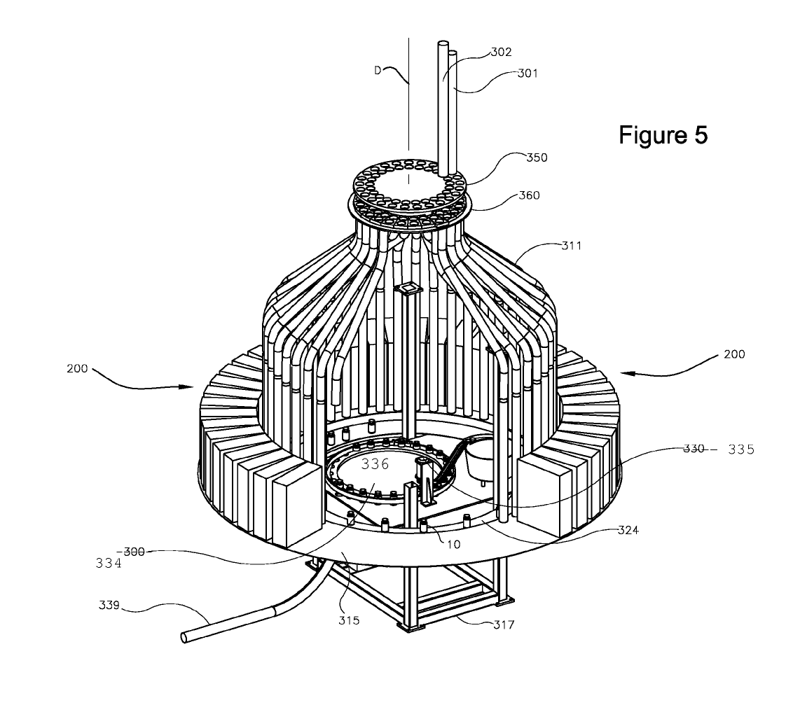

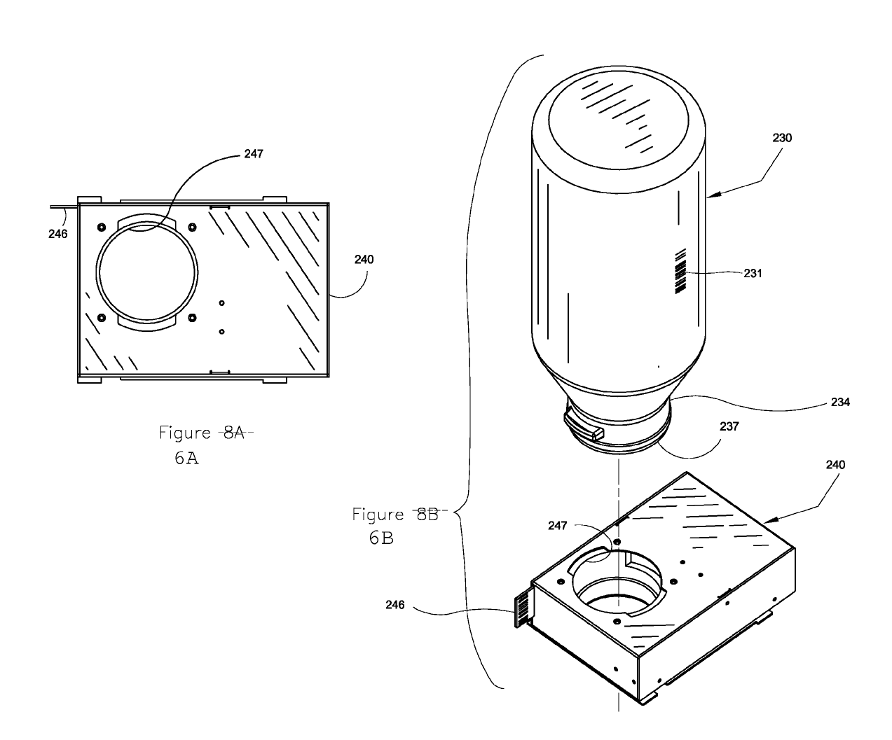

Referring now also to FIGS. 5-8, dispensing station 300 comprises an annular platform 315 supported at a convenient height above a floor by base 317 and supporting a plurality of pharmaceutical dispensing machines 200 arrayed concentrically around axis D and facing the interior of station 300. Bottle accumulator chutes 311 extend upward to dispersion wheel 350 to receive bottles 10 one at a time as system 1000 directs them to a particular dispenser 200 for filling. Once filled, bottles 10 move into the interior of station 300 to be capped, and then exit station 300 through outlet tube 339 to be urged toward verification stage 400 by pneumatic impeller 130.

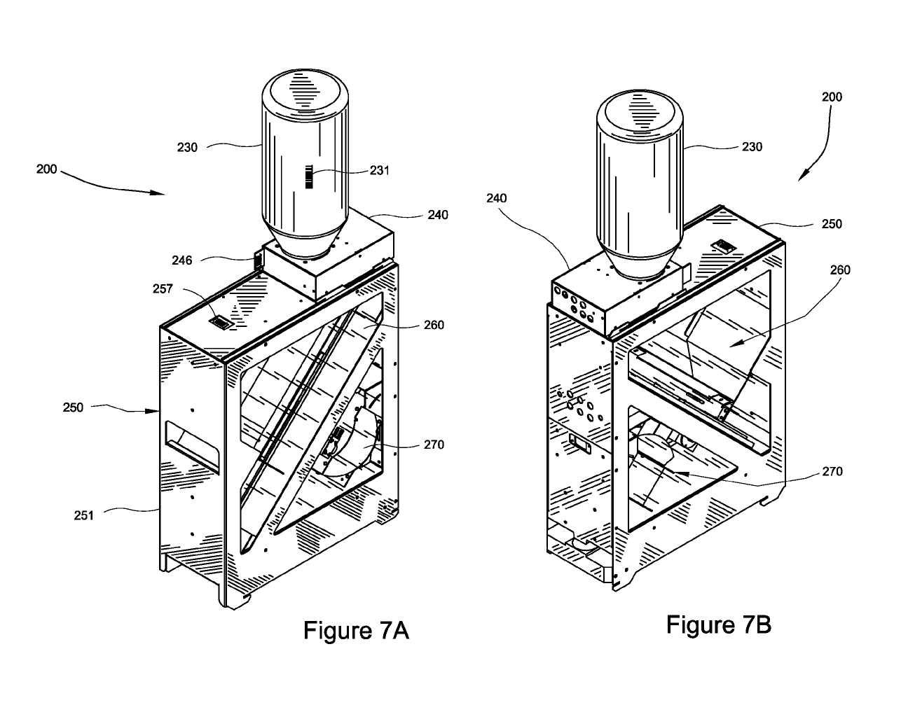

Each of dispensers 200 comprises cabinet 250 enclosing hopper 260 wherein pharmaceuticals P are staged in preparation for being counted out into bottles 10 by dispenser wheel 270. Coupled to the top of cabinet 250, bulk canister 230 is locked by lock neck device 240 and cannot be removed until system 1000 releases it. Using bar codes (best seen in FIG. 8), system 1000 assigns canister 230 a unique identifier which is matched to lock neck 240 when lock neck is installed onto canister 230. This may be performed in advance and the combined canister 230 and lock neck 240 stored in the pharmacy warehouse until needed on a dispenser 200. When so needed, an order is issued to transport a particular canister 230 and lock neck 240 to the cabinet 250 of dispenser 200 and installed (see FIGS. 7A, 7B). System 1000 further assigns a unique identifier to cabinet 250 and a location 467 on dispensing station 300 (see FIG. 9) where it subsequently will expect to have a particular pharmaceutical P available to fill bottles 10. When lock neck 240 is coupled to cabinet 250 with canister 230 on top, the installer (not shown) scans the bar codes on all three devices (lock neck 240, cabinet 250 and canister 230) and confirms that pharmaceutical P in canister 230 indeed is expected at location 467. If so, system 1000 unlocks lock neck 240 and pharmaceutical P is released into hopper 260. If the bar codes do not match, system 1000 refuses to unlock lock neck 240 and issues an alert 464.

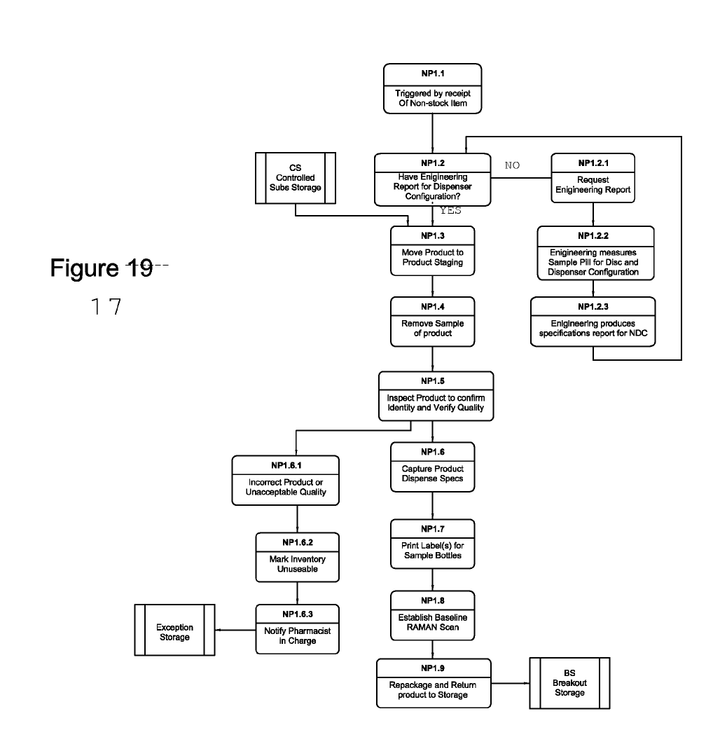

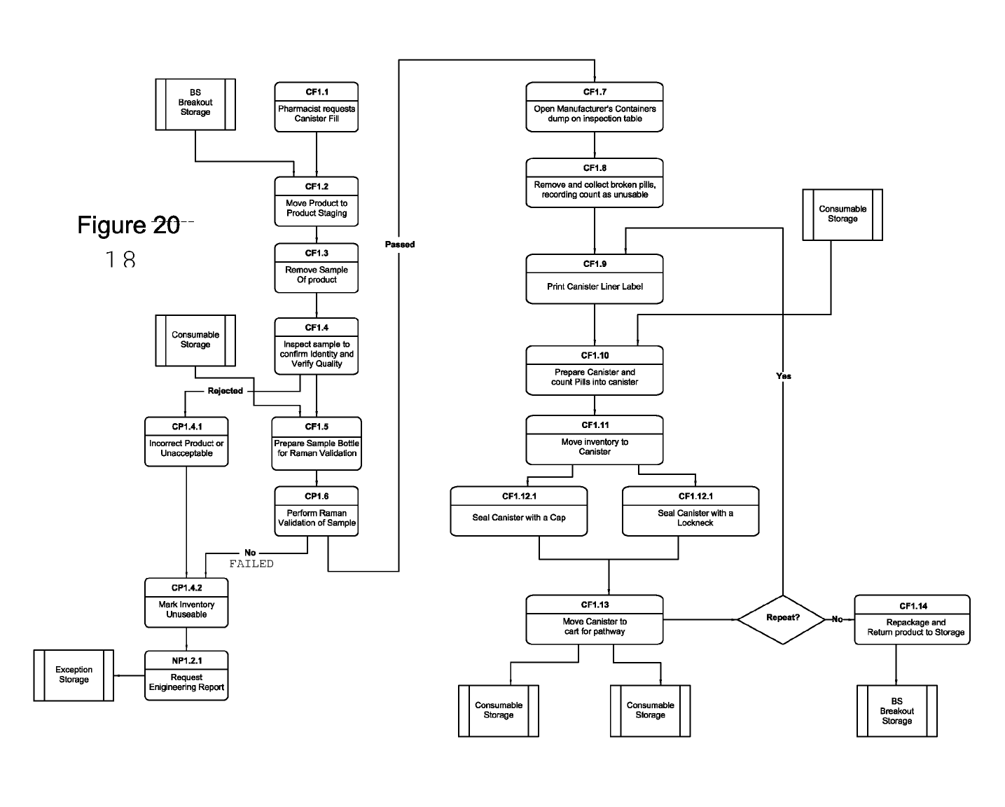

FIG. 9 comprises a graphic user interface for a warehouse manager or pharmacist (neither shown) to monitor system 1000 and dispensing station 300. Each dispenser 200 is indicated, as well as the identity 467 of the particular dispensing station 300 being monitored. The designated pharmaceutical P contained in each dispenser 200 is shown, as well as the count C of pharmaceuticals P remaining therein. When a pharmaceutical P begins to become low in a dispenser 200, system 1000 generates alert 464 and begins a procedure to replenish it. Further discussion of the canister 230 filling, lock neck 240 installation and pharmaceutical replenishment procedures appear herein below in conjunction with FIGS. 19-21.

### Pharmaceutical Dispensing

Referring now also to FIG. 8, bottles 10 arrive in dispenser accumulation chutes 311 (see also FIG. 5) and stack up until they are urged one at a time by bottle pusher 313 beneath the outfall of dispenser 200. If indicia 9 indicates bottle 10 is supposed to be filled by dispenser 200, bottle pusher 313 moves bottle 10 beneath sensor 255 to be filled. As disk 270 rotates to drop individual pills of pharmaceutical P into bottle 10, sensor 255 counts them to verify that bottle 10 receives the proper number of pills of pharmaceutical P, whereupon disk 270 stops and bottle pusher 313 extracts bottle 10 and urges it onto rotating table 324 (FIG. 5) while another bottle 10 drops into place in bottle pusher 313 to be filled at dispenser 200.

As bottles 10 leave dispensers 200, they move onto annular, moving table 324 (FIG. 5) which rotates around axis D continuously until stopped by system 1000. As bottles 10 travel around axis D, they are captured by entrance conveyor 327 and urged into capping wheel 334 which incrementally rotates to place first one bottle 10 after another under capper 335 to receive cap 50.

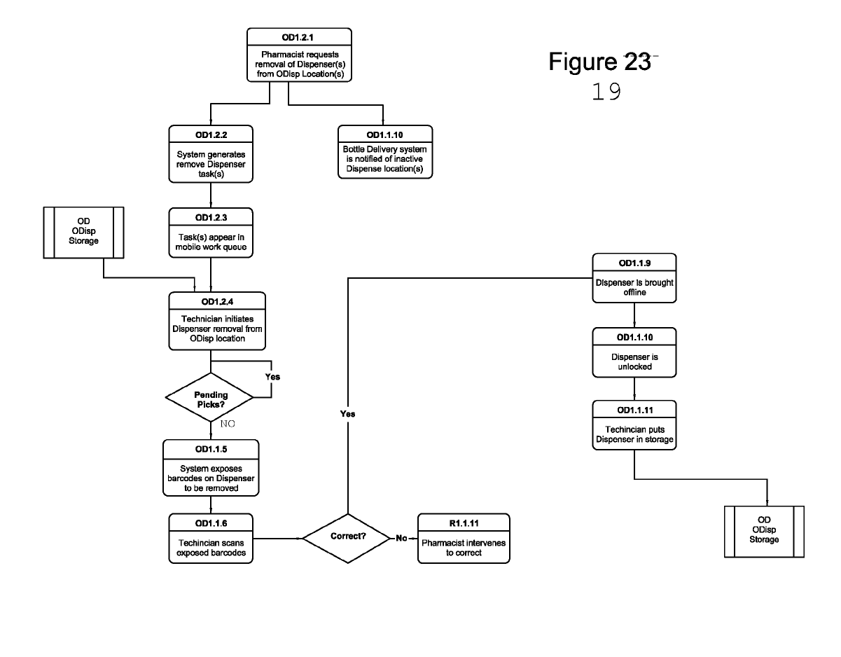

As best seen in FIG. 14, bottles 10 are captured by capping wheel 334 in notches 336 and incrementally moved into position for capping beneath capper 335. Caps 50 enter capper 335 from bowl feeder 166 on cap chute 333 and capper 335 threads them onto bottles 10, thereby sealing bottles 10 with pharmaceuticals P inside. Capping wheel 334 continues to move capped bottles 10 around its perimeter until they fall into outlet tube 339 on their way to verification stage 400. Further discussion of the operation of dispensers 200 and dispenser station 300 appears herein below in conjunction with FIGS. 22-23.

One having ordinary skill in the art will recognize that occasions may arise when the automated bottle filling process described herein above may be too cumbersome for some prescriptions, such as for very small amounts or very rarely used drugs P, and that a manual filling process may be needed. Once such manual filling is achieved, the manually filled and capped bottles 10 are fed downstream into the same verification stage 400 discussed below as is used for automatically filled bottles 10.

### Verification Stage

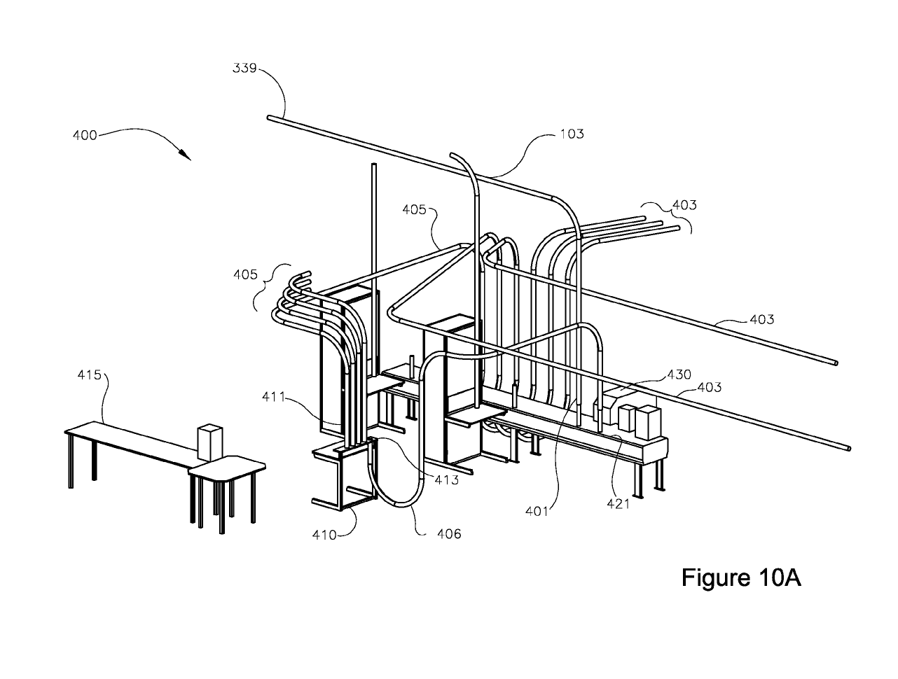

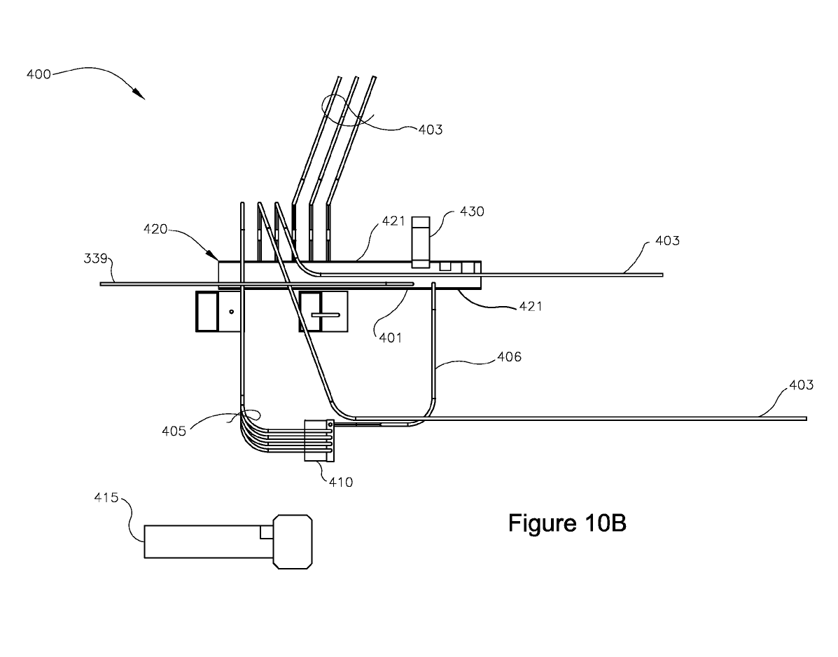

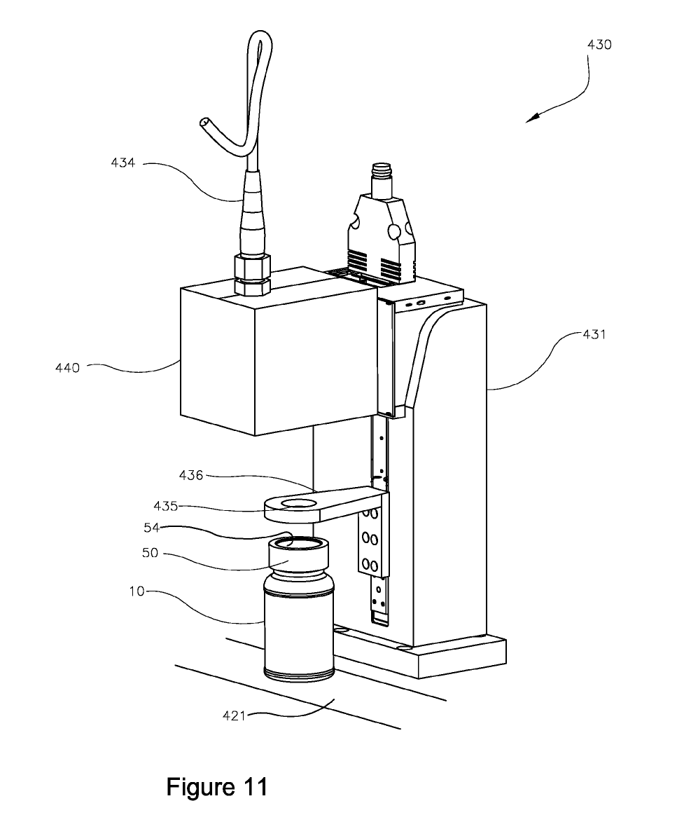

Turning now to FIGS. 10A-11, verification stage 400 comprises a process by whic contents P of each container 10 is certified to be correct according to prescription indicia 9 on labels 2. As best seen in FIGS. 1, 2, verification station 400 is positioned downstream of pharmaceutical dispensing station 300 and receives containers 10 after they have been filled from dispensers 200 and sealed. Output tube 339 of each dispensing station 300 conveys containers 10 to station 400 through tubes 103 using pneumatic propulsion. Though station 400 is depicted in the figures as corresponding one-to-one with stations 300, one having ordinary skill in the art will recognize that the number of stations 400 required to verify the results of station 300′s filling of containers 10, and the number of other sources for containers 10 (e.g. clamshell containers 40 filled with low-volume pharmaceuticals P) will dictate the number of verification stations 400 relative to other stages in system 1000.

As best seen in FIGS. 10A, 10B, each verification station 400 can feed through exit tubes 403 downstream to all sortation stations 500 in system 1000. This is because a customer having multiple prescriptions for different pharmaceuticals P, may receive containers 10 from dispensers 200 resident on several different dispensing stations 300. All such dispersed prescription containers 10 are collected by system 1000 at sortation stage 500 before they are packaged together at packaging station 600 and shipped to the customer. Accordingly, though verification station 400 likely receives incoming containers 10 from only one dispensing stage 300, it feeds containers 10 downstream through multiple sortation feed lines 403.

Also depicted in FIGS. 10A, 10B, exception station 410 comprises a location where a pharmacist (not shown) may manually inspect a container 10 to see if he can tell why it did not pass verification. The pharmacist may discover the error and re-insert container 10 into system 1000 rather than restart container 10 again at labeler 120. Exception feed lines 405 come into exception station 410 from all verification stations 400, but single exception return line 406 conveys the low volume of returned containers 10 back into verification station 400 nearest exception station 410.

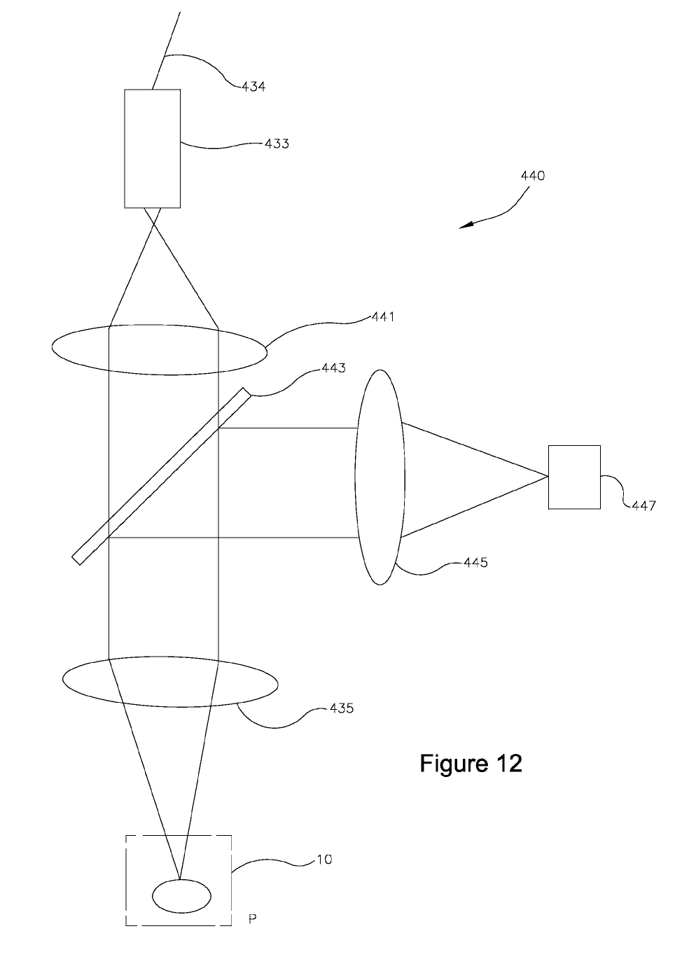

Disposed at one end of station 410, laser spectral analysis machine 440 and autofocus device 430 comprise means by which the content of each container 10 may be verified. This stage thus provides a final security confirmation and method by which errors in prescriptions may be minimized. As also shown in FIGS. 11, 12, laser machine 440 peers into the top of container 10 through transparent window 54 in cap 50 and focuses on pharmaceutical P using autofocus device 430 discussed below.

Referring now also to FIGS. 12, 14, system 1000 employs Ramon spectroscopy techniques to confirm that the content of container 10 is what is expected to be there. Container 10 is scanned to match its contents P with a prescription record from system 1000′s database which has tracked container 10 since it was labeled at labeler 120. Ramon spectroscopy measures minute quantities of pharmaceutical P back-scattered in a small cloud inside container 10 by laser 440. Each pharmaceutical P has a unique, spectral signature 454 of the elements it contains. Spectral analysis (Ramon technique) proves the best means for close focus detection and determination of such spectral signature. By comparing the spectral analysis 454 of the contents of container 10 to a library of known spectral signatures 453 of pharmaceuticals P in bottle 10, and performing a mathematical analysis to determine if they are the same, system 1000 can pass or fail the contents of container 10.

If an error is detected, or the spectral analysis cannot confirm identical pharmaceuticals P in container 10, an alert issues and the screen shown in FIG. 14 displays data associated with the error. A pharmacist at exception station 410 may review the information visually by consulting his monitoring screen depicted in FIG. 14. Therein, a second means of verifying pharmaceuticals P comprises a visual inspection of the actual contents 456 with a library image 455 of the expected pharmaceutical P. In most cases where the spectral analysis detected an error, the visual comparison will be obvious, and container 10 must be rejected. Container 10 will be discarded at rejection table 415 to be emptied and destroyed, and a new bottle 10 will begin its journey through system 1000 at labeler 120. Should the pharmacist believe, however, after inspection of his screen in FIG. 14, that the pharmaceuticals P are the same, he can reinsert container 10 into system 1000 to run through verification station 400 a second time.

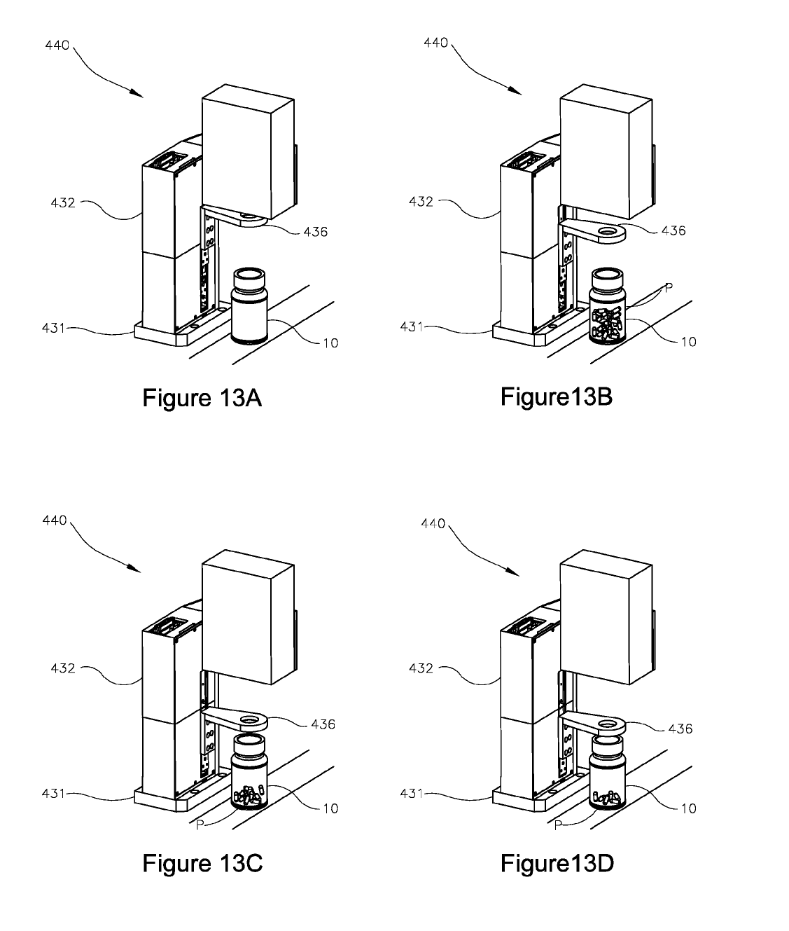

FIGS. 13A-13D depict the autofocus feature of the present invention in operation. A problem can arise in focal acuity due to different levels of pharmaceutical P within bottle 10. Particularly at low levels, where some pills may not even be directly beneath focusing lens 435, leading system 1000 to believe bottle 10 is empty. By directing laser 440 at an angle to focus on the corner of bottle 10 between bottom 20 and walls 11, and then spinning bottle 10 on its axis, laser 440 can detect even one pill inside bottle 10.

A further focus problem arises when bottle 10 is full or nearly empty. By focusing on the center of bottle 10, laser 440 may not get the best reading for visual or spectral analysis of pharmaceuticals P. The autofocus device of FIGS. 13A-13D moves lens carriage 436 upward (FIG. 13B) when bottle 10 is full, and downward (FIGS. 13C, 13D) as less and less pharmaceutical P is in bottle 10. This autofocus allows use of a narrow depth of field and more precise analysis of the backscatter and visual images of pharmaceuticals P.

### Flow Charts and Schematics of Operations

Turning now to FIGS. 15-18, system 1000 is procedurally interconnected between its receiving department R1.2, where pharmaceuticals P, among other shipments (not shown) arrive and packaging and shipping zone 600 where filled prescriptions in containers 10 are packaged and sent by common carrier (not shown) to customers (not shown). Products P and other materials used in system 1000 arrive at shipping R1.2 and immediately are assigned a bar code (not shown) by which they are tracked and accounted for throughout system 1000. Hereinafter, the discussion will follow pharmaceuticals P without regard to other shipments arriving at shipping department R1.2.

Initially, each shipment of pharmaceuticals P are contrasted by system 1000 (through its operating system-see FIG. 14) to purchase orders and special requests of expected deliveries R1.2. If a given shipment is not expected R1.3, either by a pre-existing purchase order or otherwise, it is rejected and returned unopened to the shipper or manufacturer (neither shown). Where a shipment is not the subject of a purchase order but it is expected, it is assigned a purchase order R1.3.1 and forwarded to storage R1.4.

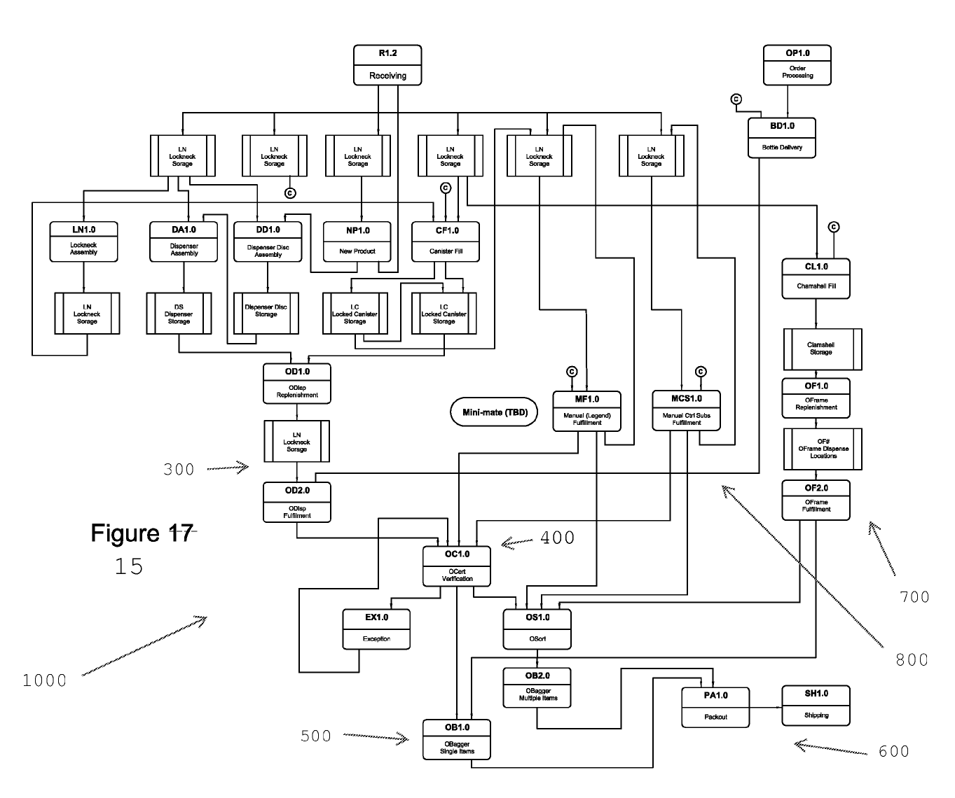

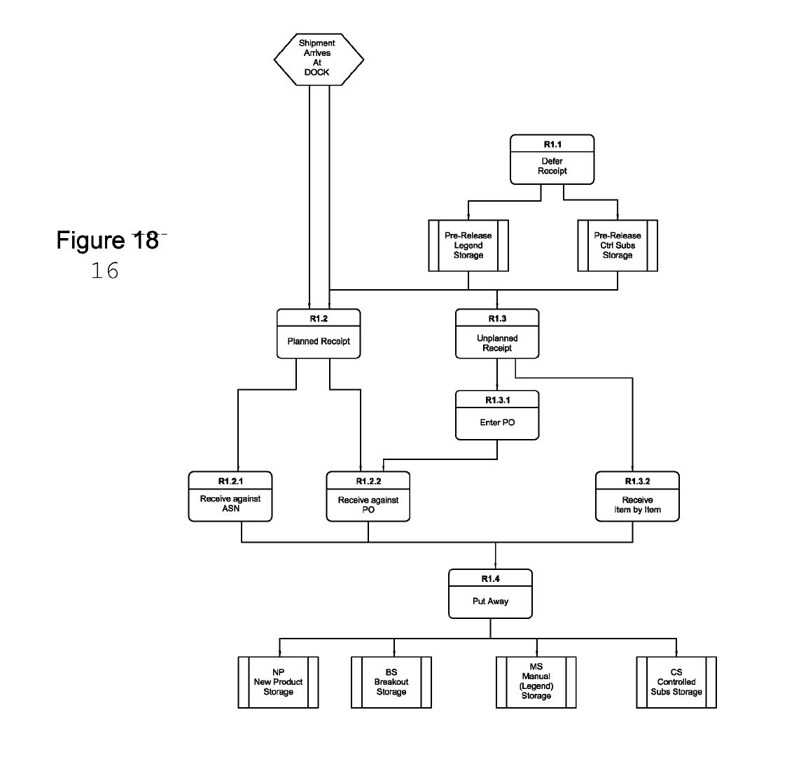

At storage station R1.4, each shipment is determined to be either a new product NP1..1 or a re-supply of previously used products. For new products, the procedure shown in FIG. 17 catalogs the pharmaceutical, including obtaining a sample NP1.4 thereof and contrasted NP1.5 to known product identities and either rejected NP1.6.1 or forwarded for use. In the latter case, pharmaceutical P must be spectrally analyzed NP1.8 for a baseline reading and then forwarded NP1.9 to breakout storage for subsequent use in system 1000.

As best seen in FIG. 18, pharmaceuticals P are prepared for use in dispenser 200 by first loading them CF1.1 into canisters 230 and sealed CF1.13. To do so, each manufacturer's container is dumped CF1.8 onto a table and inspected. Broken pills are removed CF1.8, a liner is labeled CF1.9 and inserted into canister 230 and pharmaceuticals P counted into canister 230 so that system 1000 knows exactly how many pills P are in each canister 230. Canisters 230 then are moved back to breakout storage CP1.13 either sealed or locked with a lock neck CP1.12.1, as directed by system 1000.

When a dispenser 200 requires replenishment of its supply of pharmaceuticals P, as determined by a cumulative count C (FIG. 14), system 1000 issues a canister 230 replacement order and sends a technician (not shown) to pick up another supply, transport it to the dispenser needing replenishment, and to change out one canister 230 with another. At each step, the technician scans bar codes on canister 230, lock neck 240, dispenser 200 and the location of dispenser 200 on dispenser station 300. Only when all checks have been performed and are in accordance with instructions from system 1000 can lock neck 240 be opened by system 1000 and pharmaceuticals P released into hopper 260 of dispenser 200 so that dispenser 200 may again be brought online to dispense pharmaceuticals P into containers 10, as discussed herein above.

FIGS. 22 and 23 describe the steps in the dispensing process at dispenser 200. Each bottle 10 arrives OD2.1.1 at dispensing station 300, whereupon dispersion wheel 350 scans OD2.1.2 its label 2 to determine which dispenser 200 to which to direct it, then disperses OD2.1.3 bottle 10 to the appropriate dispenser 200 location through chutes 311. When bottle 10 arrives OD2.2.1 at dispenser 200, it is scanned again OD2.2.2 to verify it is at the correct dispenser 200 and rejected OD2.2.3 if not. If it is at the correct location, bottle 10 then is filled as described above and delivered out of dispenser 200 for capping and forwarding to verification stage 400.

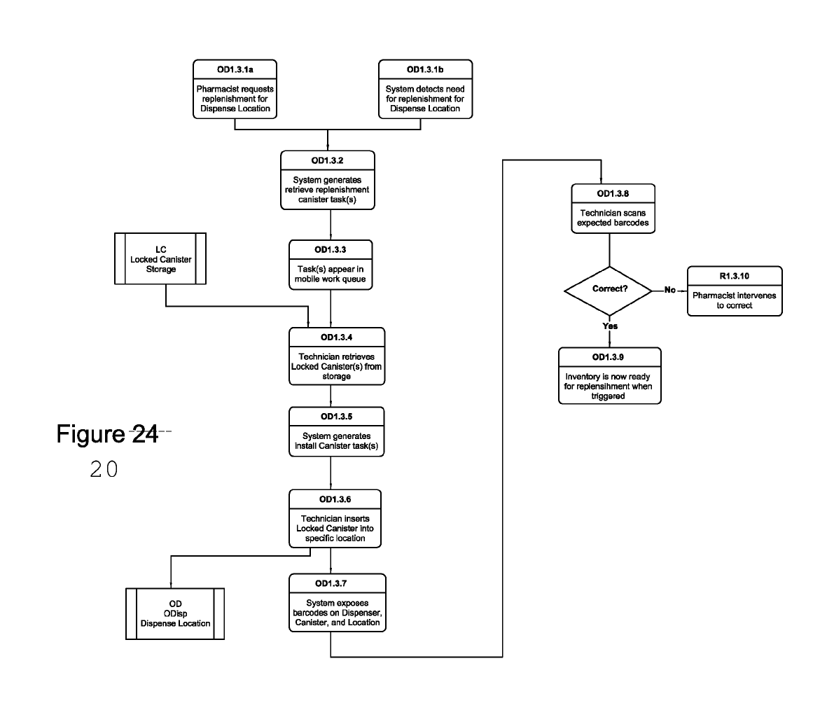

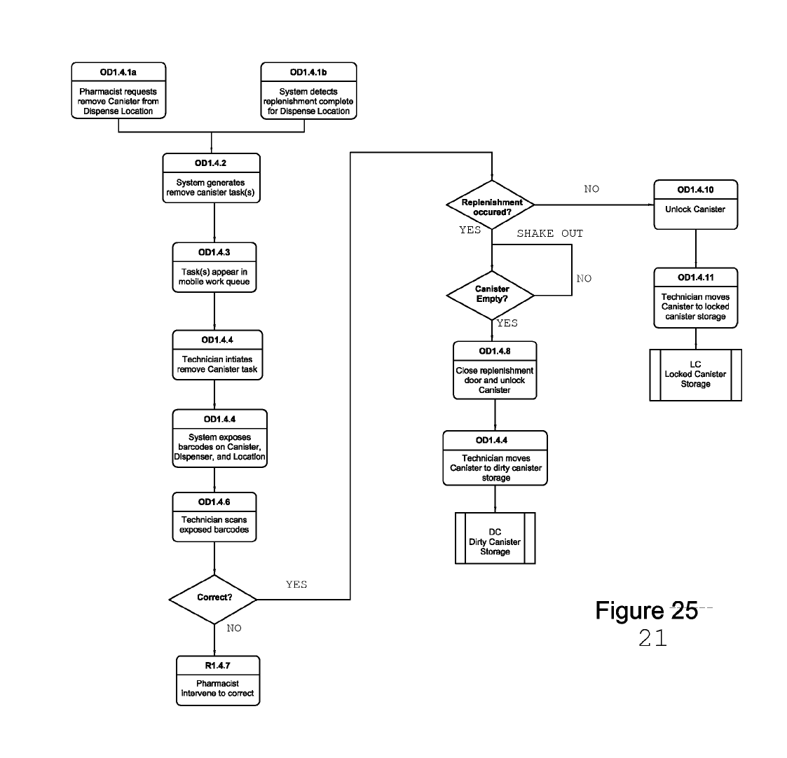

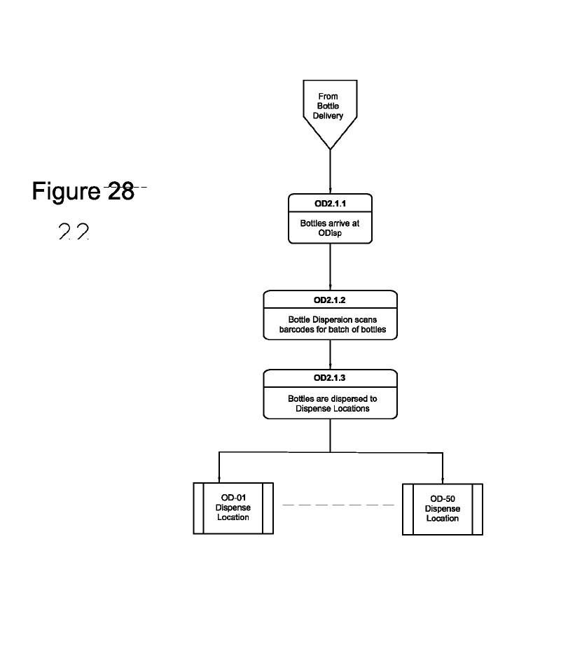

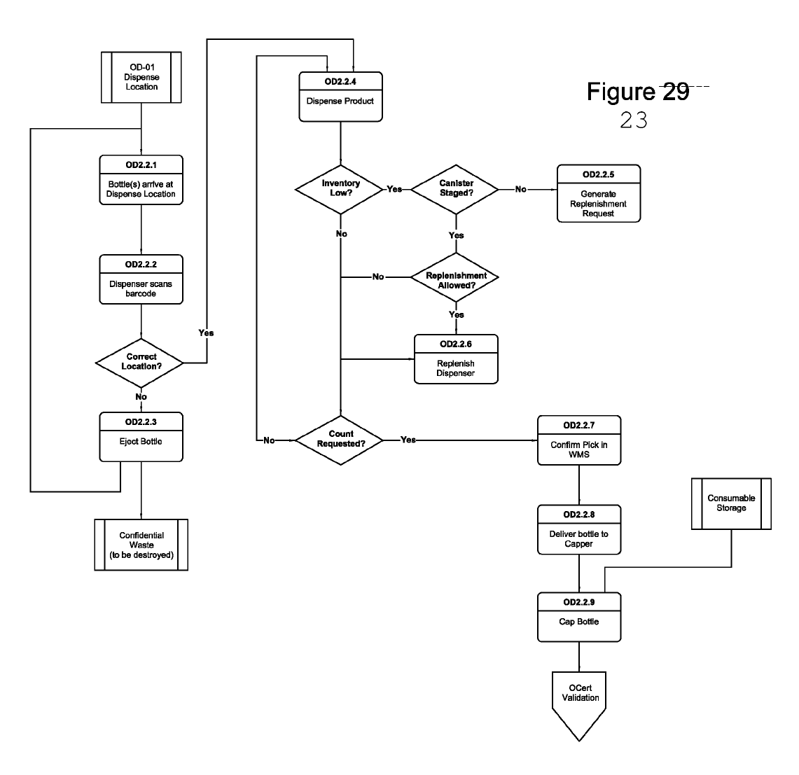

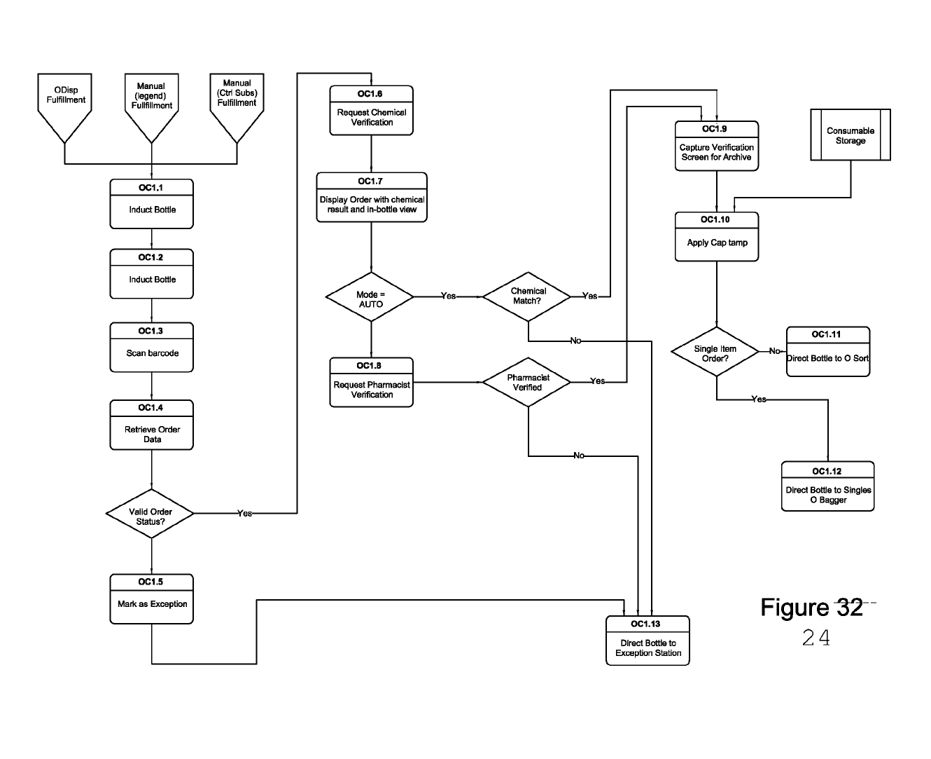

FIG. 24 shows the steps by which verification station 400 analyzes contents P of bottle 10 arriving from dispensing station 300. Again, bar code 9 is scanned to determine what contents P are supposed to be in bottle 10, and visual and Ramon spectrographic 0C1.6 scans are obtained and compared with library values, the results being displayed 0C1.7 and captured for archives 0C1.9 before a tamp (not shown) is applied to cap 50 to protect contents P from deterioration from light. Bottles 10 then are forwarded to sortition stage 500 for collating with other bottles 10 or containers 40 from inventory 700 for a given customer, then packaged and shipped at station 600.

Thus, automated prescription filling system 1000 maintains security of pharmaceuticals P from the moment they are received through dispensing, sorting, bagging and shipping to the customer. Removed from manufacturers' shipping containers as early as possible and transferred to locked canisters 230 until release into dispensers 200, pharmaceuticals P prove much more secure that otherwise. Bottles labeled with prescription information progress through dispensing of pharmaceuticals P to automated verification system 400 where they may be confirmed without re-opening bottles 10. System 1000 can detect errors and automatically restart a prescription if an error occurs.

Notably and importantly, each station 200, 300, 400, 500 and 600 operates independently of the others, scanning bar codes 9 for each bottle and checking with system 1000 as to the propriety and accuracy of its arrival and the processing that is to be performed before proceeding. This prevents mishaps which might occur between stations from causing errors in prescription fillings.

While the invention has been particularly shown and described with reference to preferred and alternate embodiments, it will be understood by those skilled in the art that various changes in form and detail may be made therein without departing from the spirit and scope of the invention. For example, though dispensing station 300 and dispensers 200 have been presented herein in the context of prescription filling of pharmaceuticals, they easily could be adapted to dispense any inventory of small objects, such as screws, nuts or other fasteners. Container 10 has been described as a bottle having dimensions convenient to the described pharmaceutical prescription application, but it could be considerably larger or smaller as required, either in similar pharmaceutical prescription filling systems or other applications, and it could be a container 10 having other shapes and characteristics which still cooperates with container transport system tubes 100 to move between stations 300, 400, 500 and 600.

I claim:

1. A pharmaceutical handling and security system for automated prescription filling, said prescriptions being a specified quantity of pharmaceuticals ordered by a physician for each of a plurality of patients, the handling and security system comprising
a plurality of standardized containers adapted to receive selected quantities of pharmaceuticals according to said prescriptions;
label means for labeling each of said standardized containers, the label means bearing machine-readable indicia of a patient's prescription;
bulk pharmaceutical storage means for storing bulk pharmaceuticals prior to dispensing them into said containers;
indicia reading means for reading said machine-readable indicia on each container;
dispensing means for dispensing pharmaceuticals into said containers; and
sortition means for sorting a plurality of containers for said patient together for packaging and shipping to said patient.

2. The pharmaceutical handling and security system according to claim 1 wherein said label means comprises
a paper label disposed on said container and bearing a bar code as said machine-readable indicia, said paper label being applied to each of said standardized containers before said dispensing means dispenses said pharmaceuticals into said containers.

3. The pharmaceutical handling and security system according to claim 1 wherein said bulk storage means comprises
a plurality of canisters adapted to contain a quantity of pharmaceuticals, each of said plurality of canisters having
a cylindrical canister body having a longitudinal canister axis extending between a canister bottom and a canister top and surrounding a canister interior;
a canister mouth disposed at said canister top and communicating with said canister interior;
a canister bar code disposed on said canister and adapted to identify and distinguish said canister from other ones of said plurality of canisters; and
sealing means disposed on said canister mouth for sealing and securing said canister interior.

4. The pharmaceutical handling and security system according to claim 3 wherein said sealing means comprises
a plurality of lock necks, each one of said plurality of lock necks adapted to surround and seal said canister mouth, each lock neck having
a lock neck body surrounding and defining a lock neck throat adapted to receive a canister mouth;
a locking gate disposed transverse said throat and adapted to articulate between a closed position sealing said canister mouth and an open position;
a machine-operable solenoid adapted to cause said locking gate to articulate between said closed position and said open position; and
a lock neck bar code disposed on said lock neck and adapted to identify and distinguish said lock neck from other lock necks utilized on said pharmaceutical handling and security system.

5. The pharmaceutical handling and security system according to claim 1 wherein said dispensing means comprise
a plurality of dispensers arrayed around an annular platform, each of said plurality of dispensers having
a dispenser cabinet having a dispenser interior containing
a hopper adapted to contain a quantity of pharmaceuticals ready for dispensing into said containers;
a dispensing wheel coupled to said hopper and adapted to count out individual pharmaceuticals into said containers in accordance with said machine readable indicia on said label means;
a dispenser input port adapted to receive securely said bulk storage means for replenishing said quantity of pharmaceuticals within said hopper; and
a first sensor means for reading said machine-readable indicia; and
a dispersion wheel disposed above said plurality of dispensers and adapted to direct each of said containers to one of said plurality of dispensers in accordance with said machine-readable indicia on sail label means.

6. The pharmaceutical handling and security system according to claim 1 wherein said sortition means comprises
a conveyor adapted to collate a plurality of said containers together for one patient in accordance with said machine-readable indicia.

7. The pharmaceutical handling and security system according to claim 1 and further comprising
a pneumatic conduit transport system coupled to and adapted to transport said containers between said labeling means, said dispensing means and said sortition means.

8. The pharmaceutical handling and security system according to claim 1 and further comprising
content verification means for verifying the contents of said containers after filling.

9. The pharmaceutical handling and security system according to claim 8 wherein said content verification means comprises
a verification station adapted to intercept said containers after said dispenser means has dispensed said pharmaceuticals into said container, said station having
a laser disposed above a conveyor and adapted to obtain a spectral signature of said pharmaceuticals within said container; and
a camera adapted to obtain a visual image of said pharmaceuticals within said container;
a controller operable to control said laser, said controller having
a library of known spectral signatures of pharmaceuticals for comparison with said spectral signature of said pharmaceuticals within said container;
a library of know visual images of pharmaceuticals for comparison with said visual images of said pharmaceuticals for comparison with said pharmaceuticals within said container; and
user interface means coupled to said controller for providing graphical comparisons of said spectral signatures and said visual images.

10. An improved method of managing a pharmacy, said pharmacy having automated pharmaceutical dispensing, sorting and packaging systems for providing high-volume prescription filling services, the method comprising
providing a handling and security system having
a plurality of standardized containers adapted to receive selected quantities of pharmaceuticals according to said prescriptions;
label means for labeling each of said standardized containers, the label means bearing machine-readable indicia of a patient's prescription;
bulk pharmaceutical storage means for storing bulk pharmaceuticals prior to dispensing them into said containers;
indicia reading means for reading said machine-readable indicia on each container;
dispensing means for dispensing pharmaceuticals into said containers;
content verification means for verifying the contents of said containers after filling; and
sortition means for sorting a plurality of containers for said patient together for packaging and shipping to said patient; then
(a) causing the containers to be directed to said dispensing means according to said machine-readable indicia; then
(b) causing said dispensing means to dispense a quantity of said pharmaceuticals into said container according to said machine-readable indicia; then
(c) directing said containers to said content verification means for certification of accuracy of said pharmaceuticals within said container according to said machine-readable indicia; then
(d) directing said container to said sortition means for collation with others of said plurality of containers containing pharmaceuticals for said patient; then
(e) repeating steps (a)-(d), inclusive, for each additional container.
